(19) United States
(12) Patent Application Publication
(10) Pub. No.: US 2026/0217641 A1
(43) Pub. Date: Jul. 30, 2026

(54) FLUORINATED FLUORINE-CONTAINING COMPOUND, AND FLUORINE-CONTAINING FLUOROPOLYMER AND SURFACE TREATMENT AGENT USING SAME

(71) Applicant: AGC Inc., Tokyo (JP)

(72) Inventors: Makoto KONAGAYA, Chigasaki-shi (JP); Yoshiharu AMANO, Chigasaki-shi (JP); Ryo HIRABAYASHI, Chigasaki-shi (JP); Taiki HOSHINO, Tokyo (JP)

(73) Assignee: AGC Inc., Tokyo (JP)

(21) Appl. No.: 19/573,109

(22) Filed: Mar. 20, 2026

Related U.S. Application Data
(63) Continuation of application No. PCT/JP2024/033734, filed on Sep. 20, 2024.

(30) Foreign Application Priority Data
Sep. 22, 2023 (JP) 2023-159062

(51) Int. Cl. C07C 69/86; C07C 235/38; C07C 271/54; C07C 275/34; C08F 120/68; C08F 120/70; C09D 133/16; C09D 133/24

(52) U.S. Cl. CPC C07C 69/86; C07C 235/38; C07C 271/54; C07C 275/34; C08F 120/68; C08F 120/70; C09D 133/16; C09D 133/24

(57) ABSTRACT

A fluorine-containing compound represented by the following formula; a fluorine-containing polymer having a structural unit formed by the fluorine-containing compound; and a surface treatment agent containing the fluorine-containing polymer and a solvent. In the formula, X is an oxygen atom, a sulfur atom, or a linking group represented by a predetermined formula, R₁ is a phenyl group in which at least one hydrogen atom is substituted with a trifluoromethoxy group, and each of R₂, R₃, and R₄ independently represents a hydrogen atom or an alkyl group having 1 to 4 carbon atoms.

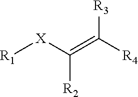

This application is a continuation application of International Application No. PCT/JP2024/033734, filed on Sep. 20, 2024, which claims the benefit of priority of Japanese Patent Application No. 2023-159062, filed on Sep. 22, 2023, the contents of which are incorporated herein by reference.

### TECHNICAL FIELD

The present disclosure relates to a fluorine-containing compound, a fluorine-containing polymer, and a surface treatment agent using the same.

### BACKGROUND ART

Conventionally, compositions containing fluorine-containing polymers and solvents have been known as waterproof and moisture-proof coating agents. Further, it has been known that the above fluorine-containing polymers may contain structural units formed by (meth)acrylic acid esters and the like having phenyl groups (see, for example, Patent Document 1).

### CITATION LIST

### Patent Document

Patent Document 1: Japanese Patent No. 6670615

### SUMMARY OF INVENTION

### Technical Problem

It has been known that compounds containing perfluoroalkyl groups generate perfluorocarboxylic acids and the like during the decomposition process.

Among perfluorocarboxylic acids and the like, since perfluorooctanesulfonic acid (PFOS) and perfluorooctanoic acid (PFOA) are highly chemically stable but resistant to degradation in the environment and also known to have a high potential for bioaccumulation and environmental accumulation, various international scientific discussions regarding target values and standards therefor have taken place, and they are becoming substances subject to restrictions in various countries.

On the other hand, since perfluorohexanoic acid (PFHxA) and perfluorocarboxylic acids with even shorter carbon chains have been confirmed to have significantly lower biotoxicity and bioaccumulation potential as compared with PFOS and PFOA, perfluoroalkyl group-containing compounds whose decomposition products are PFHxA or perfluorocarboxylic acids with even shorter carbon chains are widely used as alternative technologies.

However, even with these alternative compounds, perfluorocarboxylic acid as the decomposition product remains chemically stable, and when continued to be released into the environment, it could accumulate in the environment over a long period of time, and remains to be a concern for adversely affecting human bodies and the environment. Therefore, there is a demand for new alternative compounds.

Meanwhile, compounds containing trifluoromethoxy groups are known to readily decompose in the environment (for example, in an environment above −20° C.) because the CF₃OH expected to be generated during the decomposition process is a highly unstable compound.

Accordingly, an object of the present disclosure is to provide a novel fluorine-containing compound that has a trifluoromethoxy group and can form a polymer with excellent water repellency and oil repellency.

Another object of the present disclosure is to provide a fluorine-containing polymer and a surface treatment agent.

### Solution to Problem

As a result of intensive studies in order to solve the above problems, the inventors of the present disclosure have found that the problems can be solved by the following configuration.
[1] A fluorine-containing compound represented by the following formula (M):

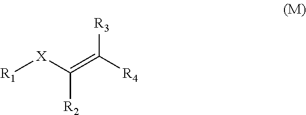

In the formula (M), X is an oxygen atom, a sulfur atom, or a linking group represented by the following formula (A); R₁ is a phenyl group in which at least one hydrogen atom is substituted with a trifluoromethoxy group; and each of R₂, R₃, and R₄ independently represents a hydrogen atom or an alkyl group having 1 to 4 carbon atoms.

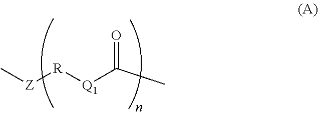

In the formula (A), n is an integer from 0 to 8; Q₁ is an oxygen atom or a divalent group represented by —NH—; R is a linear or branched alkylene group having 1 to 4 carbon atoms, or a phenylene group; and Y is any one of linking groups represented by the following formulas (y1) to (y4).

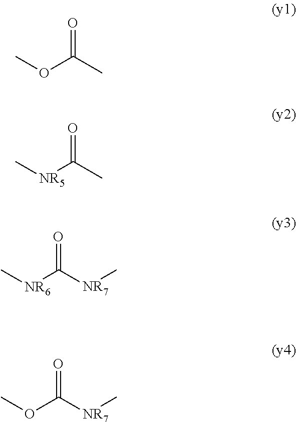

In the formulas (y2) to (y4), each of R₅ and R₆ independently represents a hydrogen atom, an alkyl group having 1 to 4 carbon atoms, or a group represented by the following formula (B); and each of R₇ and R₈ independently represents a hydrogen atom or an alkyl group having 1 to 4 carbon atoms.

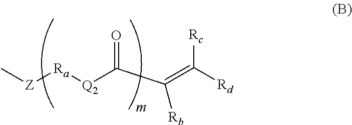

In the formula (B), m is an integer from 0 to 8; Q₂ is an oxygen atom or a divalent group represented by —NH—; Rₐ is a linear or branched alkylene group having 1 to 4 carbon atoms, or a phenylene group; each of R_{b}, R_{c}, and Rₐ independently represents a hydrogen atom or an alkyl group having 1 to 4 carbon atoms; and Z is a linking group represented by the following formula (z1) or (z2).

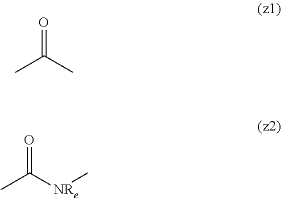

In the formula (z2), Rₑ is a hydrogen atom or an alkyl group having 1 to 4 carbon atoms.
[2] The fluorine-containing compound according to [1], wherein in the aforementioned R₁, the aforementioned trifluoromethoxy group is bonded to a para position of the aforementioned phenyl group.
[3] The fluorine-containing compound according to [1] or [2], wherein in the aforementioned formula (M), X is a linking group represented by the aforementioned formula (A).
[4] The fluorine-containing compound according to [3], wherein the aforementioned Y in the aforementioned formula (A) is a linking group represented by the aforementioned formula (y1) or (y2).
[5] A fluorine-containing polymer having a structural unit formed by the fluorine-containing compound according to any one of [1] to [4].
[6] A surface treatment agent containing the fluorine-containing polymer according to [5] and a solvent.

### Advantageous Effects of Invention

According to the present disclosure, it is possible to provide a novel fluorine-containing compound that has a trifluoromethoxy group and can form a polymer with excellent water repellency and oil repellency.

The present disclosure can also provide a fluorine-containing polymer and a surface treatment agent.

### DESCRIPTION OF EMBODIMENTS

The present disclosure will be described in detail below.

The explanation for the constituent elements described below may be based on representative embodiments of the present disclosure, but the present disclosure is not limited to such embodiments.

In the present specification, a numerical range expressed using “to” means a range including the numerical values described before and after the “to”.

In the present specification, each component can be used alone, or two or more types thereof can be used in combination.

In the present specification, when two or more types of a component are used in combination, the “content” of that component means the total content of those two or more types, unless otherwise specified.

In the present specification, the production method of each component is not particularly limited unless otherwise specified. Examples thereof include conventionally known methods.

In the present specification, (meth)acrylic acid refers to acrylic acid or methacrylic acid.

In the present specification, with regard to the polymer obtained from the fluorine-containing compound of the present disclosure, the fluorine-containing polymer of the present disclosure, or the surface treatment agent of the present disclosure, when the water repellency and oil repellency thereof are further improved, it is also referred to as “the effects of the present disclosure are further improved.”

### [Fluorine-Containing Compound of the Present Disclosure]

The fluorine-containing compound of the present disclosure will be described below.

The fluorine-containing compound of the present disclosure is a fluorine-containing compound represented by the following formula (M).

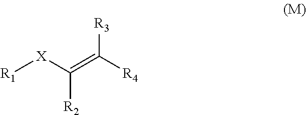

In the formula (M), X represents an oxygen atom, a sulfur atom, or a linking group represented by the formula (A) described below; R₁ represents a phenyl group in which at least one hydrogen atom is substituted with a trifluoromethoxy group; and each of R₂, R₃, and R₄ independently represents a hydrogen atom or an alkyl group having 1 to 4 carbon atoms. R₂ is preferably a hydrogen atom or a methyl group. R₃ and R₄ are preferably hydrogen atoms.

Even if the fluorine-containing compound of the present disclosure or the fluorine-containing polymer of the present disclosure is unexpectedly released into the environment, since CF₃OH which can be generated originating from the trifluoromethoxy group upon decomposition is an unstable compound and readily decomposes in the environment (for example, in an environment above −20° C.), it is possible to significantly reduce the risk of it remaining in the environment and traveling long distances. It is believed that the fluorine-containing polymer of the present disclosure and the like can reduce environmental burden.

In the present disclosure, in the formula (M), X is an oxygen atom, a sulfur atom, or a linking group represented by the following formula (A). From the viewpoints of further improving the effects of the present disclosure and not impairing water repellency and oil repellency, X is preferably a linking group represented by the following formula (A).

The linking group represented by the formula (A) is as follows.

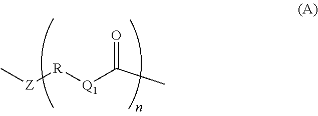

In the formula (A), n is an integer from 0 to 8. From the viewpoints of further improving the effects of the present disclosure and not impairing water repellency and oil repellency, n is preferably 0 or 1, and more preferably 0.

In the formula (A), R is a linear or branched alkylene group having 1 to 4 carbon atoms, or a phenylene group.

Examples of the linear or branched alkylene group having 1 to 4 carbon atoms include a linear alkylene group such as a methylene group, an ethylene group, a trimethylene group, and a tetramethylene group; and a branched alkylene group such as an isopropylene group and an isobutylene group.

When the above n is 1 or greater, from the viewpoints of further improving the effects of the present disclosure and not impairing water repellency and oil repellency, R is preferably a linear alkylene group having 2 to 4 carbon atoms or a phenylene group, and more preferably a phenylene group.

In the formula (A), Q₁ is an oxygen atom or a divalent group represented by —NH—.

In the formula (A), Y is any one of the linking groups represented by the following formulas (y1) to (y4).

It should be noted that when incorporating the linking groups represented by the formulas (y1) to (y4) into Y in the formula (A), the formulas (y1) to (y4) can be incorporated into Y in the formula (A) either in a state as described below or by reversing them left and right, but it is preferable to incorporate the linking groups represented by the formulas (y1) to (y4) into Y in the formula (A) in the state as described below.

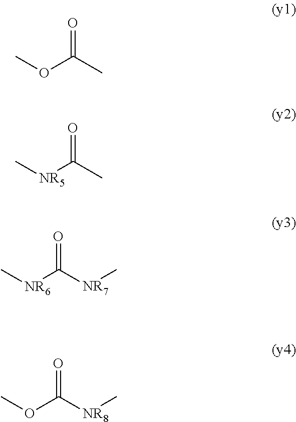

In the formulas (y2) to (y4), each of R₅ and R₆ independently represents a hydrogen atom, an alkyl group having 1 to 4 carbon atoms, or a group represented by the following formula (B).

Examples of the above alkyl group having 1 to 4 carbon atoms represented by R₅ and R₆ include a methyl group, an ethyl group, a propyl group, and a butyl group.

The group represented by the formula (B) is as follows.

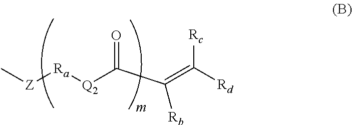

In the formula (B), m is an integer from 0 to 8.

In the formula (B), Rₐ is a linear or branched alkylene group having 1 to 4 carbon atoms, or a phenylene group. Examples of the linear or branched alkylene group having 1 to 4 carbon atoms represented by Rₐ include a linear alkylene group such as a methylene group, an ethylene group, a trimethylene group, and a tetramethylene group; and a branched alkylene group such as an isopropylene group and an isobutylene group.

In the formula (B), Q₂ is an oxygen atom or a divalent group represented by —NH—.

In the formula (B), each of R_{b}, R_{c}, and Rₐ independently represents a hydrogen atom or an alkyl group having 1 to 4 carbon atoms. Examples of the alkyl groups having 1 to 4 carbon atoms represented by R_{b}, R_{c}, and Rₐ include a methyl group, an ethyl group, a propyl group, and a butyl group.

In the formula (B), Z is a linking group represented by the following formula (z1) or (z2). It should be noted that the linking group represented by the formula (z1) or (z2) may be incorporated into Z in the formula (B) in a state described below.

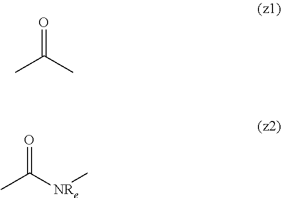

In the formula (z2), Rₑ is a hydrogen atom or an alkyl group having 1 to 4 carbon atoms. Examples of the alkyl group having 1 to 4 carbon atoms represented by Re include a methyl group, an ethyl group, a propyl group, and a butyl group.

In the formulas (y2) to (y4), each of R₇ and R₈ independently represents a hydrogen atom or an alkyl group having 1 to 4 carbon atoms. Examples of the alkyl groups having 1 to 4 carbon atoms represented by R₇ and R₈ include a methyl group, an ethyl group, a propyl group, and a butyl group.

From the viewpoint of further improving the effects of the present disclosure, Y is preferably any one of the linking groups represented by formulas (y1) to (y2), and more preferably a linking group represented by the formula (y1) or a linking group represented by the formula (y2) and in which R₅ is a hydrogen atom.

In the present disclosure, in the formula (M), R₁ is a phenyl group in which at least one hydrogen atom is substituted with a trifluoromethoxy group.

It is sufficient for R₁ as long as at least one hydrogen atom of the five hydrogen atoms included in one phenyl group is substituted with a trifluoromethoxy group. It is also possible that two or more hydrogen atoms in the above phenyl group may be substituted with trifluoromethoxy groups.

In order to exhibit the effects of the present disclosure, it is necessary for R₁ that at least one hydrogen atom of the above phenyl group is substituted with a trifluoromethoxy group.

The trifluoromethoxy group may be bonded to any of the ortho (o-), meta (m-), and para (p-) positions of the above phenyl group.

From the viewpoint of further improving the effects of the present disclosure, it is preferable that the trifluoromethoxy group be bonded to the para position of the above phenyl group in R₁.

In the present disclosure, in the formula (M), each of R₂, R₃, and R₄ independently represents a hydrogen atom or an alkyl group having 1 to 4 carbon atoms. Examples of the alkyl groups having 1 to 4 carbon atoms represented by R₂, R₃, and R₄ include a methyl group, an ethyl group, a propyl group, and a butyl group.

From the viewpoint of further improving the effects of the present disclosure, the combination of R₂, R₃, and R₄ is preferably such an embodiment that R₂, R₃, and R₄ are all hydrogen atoms, or such an embodiment that R₂ is a methyl group and R₃ and R₄ are hydrogen atoms. The most preferable embodiment is such that R₂, R₃, and R₄ are all hydrogen atoms.

From the viewpoint of further improving the effects of the present disclosure, specific examples of more preferable structures of the fluorine-containing compound of the present disclosure are shown below.

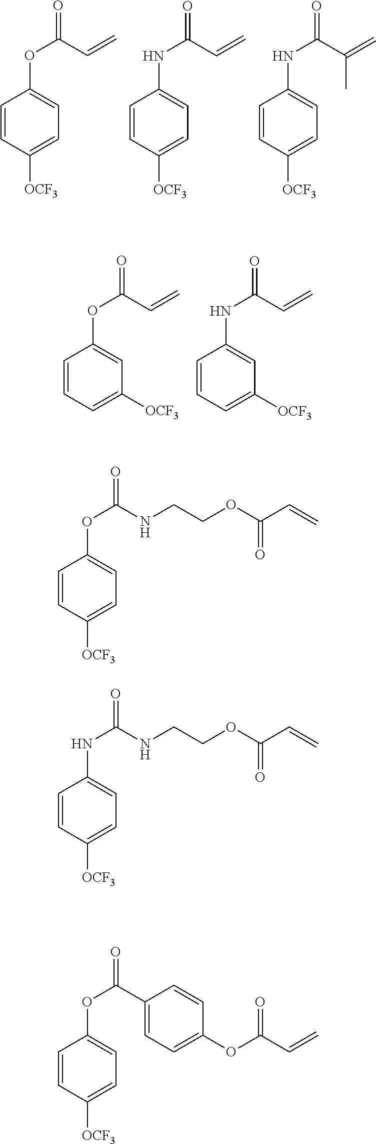

Examples of methods for synthesizing the fluorine-containing compound of the present disclosure include the following synthesis methods 1 and 2.

### —Synthesis Method 1

When the fluorine-containing compound of the present disclosure has the formula (y1) or (y2) as Y in the formula (M), examples of the Synthesis Method 1 as the corresponding synthesis method include a method of reacting trifluoromethoxyphenol, trifluoromethoxyaniline, or a precursor represented by the following formula (pre1) with (meth)acryloyl chloride in a solvent (for example, dichloromethane, methylene chloride) in the presence of a catalyst (for example, triethylamine, triethylenediamine). After the reaction, purification can be performed if appropriate.

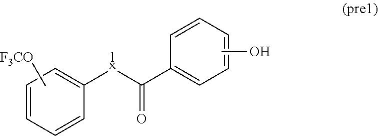

In the formula (pre1), x1 is an oxygen atom or a divalent group represented by —NH—.

### —Synthesis Method 2

When the fluorine-containing compound of the present disclosure has the formula (y3) or (y4) as Y in the formula (M), examples of the Synthesis Method 2 as the corresponding synthesis method include a method of reacting trifluoromethoxyphenol or trifluoromethoxyaniline with a (meth)acrylic acid ester having an isocyanate group, such as 2-isocyanatoethyl (meth)acrylate, in a solvent (for example, tetrahydrofuran). A catalyst (for example, N,N-diisopropylethylamine, triethylamine, triethylenediamine) may be used in the above reaction. After the reaction, purification can be performed if appropriate.

### —Applications

The fluorine-containing compound of the present disclosure can be used, for example, as a monomer for producing a polymer.

### [Fluorine-Containing Polymer of the Present Disclosure]

The fluorine-containing polymer of the present disclosure is a fluorine-containing polymer having a structural unit formed by the fluorine-containing compound of the present disclosure.

The structural unit included in the fluorine-containing polymer of the present disclosure is not particularly limited, as long as it includes a structural unit formed by the fluorine-containing compound of the present disclosure.

The structural unit formed by the fluorine-containing compound of the present disclosure has a structure in which the double bond in the above formula (M) is cleaved. The structural unit formed by the fluorine-containing compound of the present disclosure has a phenyl group derived from R₁ in the above formula (M) in which at least one hydrogen atom is substituted with a trifluoromethoxy group.

The fluorine-containing polymer of the present disclosure can include structural units formed by the fluorine-containing compound of the present disclosure, either alone or in combination of two or more types.

The structural unit included in the fluorine-containing polymer of the present disclosure may further include a structural unit (another structural unit) other than the structural unit formed by the fluorine-containing compound of the present disclosure.

The another structural unit is not particularly limited, as long as it is a structural unit formed by a monomer copolymerizable with the fluorine-containing compound of the present disclosure.

From the viewpoint of reducing the environmental burden, the fluorine-containing polymer of the present disclosure desirably does not contain a structural unit containing a perfluoroalkyl group other than a trifluoromethoxy group.

Further, from the viewpoint of maintaining water repellent performance and oil repellent performance, with respect to the total mass of all structural units included in the fluorine-containing polymer of the present disclosure, the structural unit formed by the fluorine-containing compound of the present disclosure is desirably contained in an amount of 50% by mass or more and 100% by mass or less, more preferably 80% by mass or more, and still more preferably 90% by mass or more.

The mass average molecular weight of the fluorine-containing polymer of the present disclosure is not particularly limited, but from the viewpoints of oil resistance and water resistance, the lower limit of the above mass average molecular weight is preferably 10,000 or more, and from the viewpoint of solubility in solvents, the upper limit of the above mass average molecular weight is preferably 1,000,000 or less. It should be noted that the above mass average molecular weight is a weight average molecular weight measured by gel permeation chromatography (GPC).

A method for producing the fluorine-containing polymer of the present disclosure is not particularly limited. As an example of a method for producing the fluorine-containing polymer of the present disclosure, for example, the fluorine-containing polymer of the present disclosure can be produced by polymerizing the fluorine-containing compound of the present disclosure in a solvent (for example, ethyl acetate) in the presence of a polymerization initiator (for example, dimethyl 2,2′-azobis(2-methylpropionate)) under a condition of 60 to 80° C. Further, during the polymerization, a monomer copolymerizable with the fluorine-containing compound of the present disclosure may be further used, if necessary.

The fluorine-containing polymer of the present disclosure can be used, for example, in a surface treatment agent.

### [Surface Treatment Agent of the Present Disclosure]

The surface treatment agent of the present disclosure is a surface treatment agent containing the fluorine-containing polymer of the present disclosure and a solvent. Because the surface treatment agent of the present disclosure exhibits excellent water repellency and oil repellency, it can be used as a water- and oil-repellent composition and a water- and oil-repellent agent.

The fluorine-containing polymer contained in the surface treatment agent of the present disclosure is not particularly limited, as long as it is the fluorine-containing polymer of the present disclosure.

The solvent contained in the surface treatment agent of the present disclosure is not particularly limited, as long as it is capable of dispersing and/or dissolving the fluorine-containing polymer of the present disclosure.

Examples of the solvent include a fluorine-based solvent, a hydrocarbon-based organic solvent, an ester (such as ethyl acetate and butyl acetate), and a ketone (such as acetone and methyl ethyl ketone).

Examples of the fluorine-based solvent include a hydrofluorocarbon (HFC) and a hydrofluoroether (HFE).

Specific examples of the fluorine-based solvent include m-xylene hexafluoride, p-xylene hexafluoride, CF₃CH₂CF₂CH₃, CF₃CH₂CF₂H, C₆F₁₃OCH₃, C₆F₁₃OC₂H₅, C₆F₁₃CH₂CH₃, C₃F₇OCH₃, C₃F₇OC₂H₅, C₆F₁₃H, CF₂HCF₂CH₂OCF₂CF₂H, CF₃CFHCFHCF₂CH₃, CF₃(OCF₂CF₂)ₙ(OCF₂)ₘOCF₂H, C₈F₁₇OCH₃, C₇F₁₅OCH₃, C₇F₁₃OCH₃, C₄F₉OCH₃, C₄F₉OC₂H₅, C₄F₉CH₂CH₃, CF₃CH₂OCF₂CF₂CF₂H, CF₃CF(CH₂CF₃)CF(OCH₃)CF₂CF₃, CF₂HCF₂OCH₂CF₃, and a mixture thereof.

Examples of the mixture include a mixture of CF₃ (CF₂)₃OC₂H₅ and (CF₃)₂CFCF₂OC₂H₅, which are isomers of C₄F₉OCH₂CH₃ (ethyl nonafluorobutyl ether).

It should be noted that in the above examples, the subscripts m and n each independently represent an integer from 1 to 20.

Examples of the hydrocarbon-based organic solvent include aromatic hydrocarbons (such as xylene, toluene, and ethylbenzene), alicyclic hydrocarbons (such as cyclohexane, cyclohexene, methylcyclohexane, and ethylcyclohexane), and chain aliphatic hydrocarbons (such as hexane, heptane, octane, and decane).

From the viewpoint of fully exhibiting the effects of the present disclosure, the concentration of the fluorine-containing polymer of the present disclosure contained in the surface treatment agent of the present disclosure is preferably from 0.1 to 10% by mass, and more preferably from 1 to 5% by mass, of the total amount of the surface treatment agent of the present disclosure.

The content of the solvent is not particularly limited, but from the viewpoint of handling properties and the like as a surface treatment agent, it is preferably 80% by mass or more of the total amount of the surface treatment agent of the present disclosure.

The surface treatment agent of the present disclosure can further contain an additive such as a pH adjuster, a rust inhibitor, a dye, a flame retardant, an antifoaming agent, or an antistatic agent, and a polymer or resin other than the fluorine-containing polymer of the present disclosure, as necessary, within a range that does not impair the effects of the present disclosure.

Examples of a method for producing the surface treatment agent of the present disclosure include a method of mixing the fluorine-containing polymer of the present disclosure, a solvent, and an additive that can be contained if necessary to obtain the surface treatment agent of the present disclosure.

Examples of a method for using the surface treatment agent of the present disclosure include a use method in which the surface treatment agent of the present disclosure is applied to a substrate. By the above applying process, it is possible to form a layer of the surface treatment agent on the surface of the above substrate and to improve the oil repellency of the surface of the above substrate. In addition, by the above applying process, excellent water repellency can be further imparted to the surface of the above substrate.

There are no particular limitations on the method for applying the surface treatment agent of the present disclosure to a substrate. For example, coating or the like can be mentioned.

After applying the surface treatment agent of the present disclosure to a substrate, the above solvent can be dried, for example, under a condition of 10 to 120° C.

Examples of materials for the above substrate include glass, plastics, rubber, metals, and ceramics.

Specific examples of the above substrate include household items (such as umbrellas, shoes, and bags), molded products for wet areas (such as bathroom components, washbasin components, and kitchen components), relevant exteriors of buildings (such as bridge piers, roofs, and outer walls), relevant interiors (such as floors and inner walls), housing products (such as furniture and home appliances), vehicle bodies (such as ships, airplanes, and automobiles, including exterior or interior materials), and electronic substrates.

### EXAMPLES

The present disclosure will be described in more detail with reference to the following Examples. Examples 1 to 15 are examples according to the present disclosure, and Examples 16 to 19 are Comparative Examples.

The materials, amounts used, ratios, processing details, and processing procedures shown in the following Examples can be modified as appropriate without departing from the spirit of the present disclosure. Therefore, the scope of the present disclosure should not be construed as being limited by the Examples shown below.

Hereinafter, unless otherwise specified, the terms “parts” and “%” refer to “parts by mass” and “% by mass.”

In the present specification, a fluorine-containing compound produced in Example 1 will be referred to as monomer 1, a fluorine-containing polymer produced using the monomer 1 will be referred to as fluorine-containing polymer 1, and a surface treatment agent containing the fluorine-containing polymer 1 will be referred to as surface treatment agent 1 in some cases. The same applies to other Examples.

For the compounds used as raw materials and the like in these Examples, unless otherwise specified, the above compounds were obtained from the market.

### <¹H-NMR Measurement>

A target substance to be measured (each monomer synthesized in the Examples according to the present disclosure) was dissolved in chloroform-d as a deuterated solvent so that the concentration of the target substance to be measured was adjusted to approximately 0.2% by mass. SiMe4 (tetramethylsilane) was used as the standard. The prepared solution was transferred to a ¹H-NMR measurement tube.

The measurement conditions are shown below.
Apparatus: JNM-ECZ400R/S1 (manufactured by JEOL Ltd.)
Nucleus: proton
Number of scans: 16

### Example 1

### —Synthesis of Monomer 1

9.21 g (51.7 mmol) of 4-trifluoromethoxyphenol and 78 mL of dichloromethane were mixed in a four-neck flask and immersed and cooled in ice water. After adding 7.85 g (77.5 mmol) of triethylamine dropwise, 5.61 g (62.0 mmol) of acryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 11.25 g of the desired product.

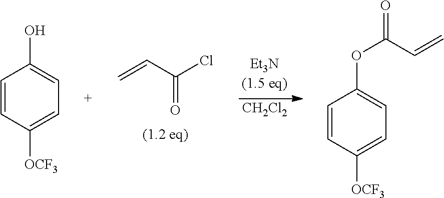

The ¹H-NMR chemical shifts of the compound obtained by the above reaction were as follows:

δ (ppm): 6.04 (dd, 1H, CH), 6.32 (dd, 1H, CH), 6.62 (dd, 1H, CH), 7.17 (m, 2H, 2CH), 7.25 (m, 2H, 2CH)

From the above ¹H-NMR results, it was confirmed that the compound obtained by the above reaction had a structure of the reaction product shown in the above reaction scheme.

### Example 2

### —Synthesis of Monomer 2

9.21 g (51.7 mmol) of 4-trifluoromethoxyphenol and 60 mL of dichloromethane were mixed in a four-neck flask and immersed and cooled in ice water. After adding 7.85 g (77.5 mmol) of triethylamine dropwise, 6.53 g (62.0 mmol) of methacryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 11.45 g of the desired product.

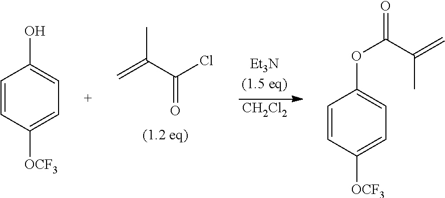

The ¹H-NMR chemical shifts of the compound obtained by the above reaction were as follows:

δ (ppm): 2.06 (m, 3H, CH₃), 5.78 (m, 1H, CH), 6.36 (m, 1H, CH), 7.16 (m, 2H, 2CH), 7.24 (m, 2H, 2CH)

From the above ¹H-NMR results, it was confirmed that the compound obtained by the above reaction had a structure of the reaction product shown in the above reaction scheme.

### Example 3

### —Synthesis of Monomer 3

8.05 g (45.4 mmol) of 4-trifluoromethoxyaniline and 80 mL of dichloromethane were mixed in a four-neck flask and immersed and cooled in ice water. After adding 5.25 g (51.9 mmol) of triethylamine dropwise, 3.92 g (43.3 mmol) of acryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 8.38 g of the desired product.

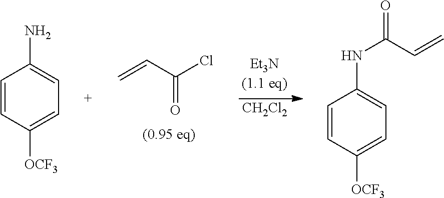

The ¹H-NMR chemical shifts of the compound obtained by the above reaction were as follows:

δ (ppm): 5.78 (dd, 1H, CH), 6.26 (dd, 1H, CH), 6.44 (dd, 1H, CH), 7.18 (m, 2H, 2CH), 7.63 (m, 3H, 2CH, NH)

From the above ¹H-NMR results, it was confirmed that the compound obtained by the above reaction had a structure of the reaction product shown in the above reaction scheme.

### Example 4

### —Synthesis of Monomer 4

9.65 g (54.5 mmol) of 4-trifluoromethoxyaniline and 96 mL of dichloromethane were mixed in a four-neck flask and immersed and cooled in ice water. After adding 5.78 g (57.1 mmol) of triethylamine dropwise, 5.43 g (51.9 mmol) of methacryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 11.50 g of the desired product.

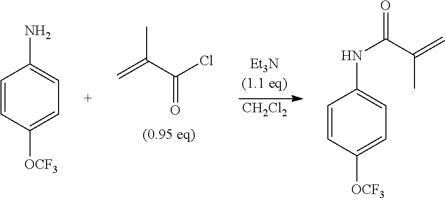

The ¹H-NMR chemical shifts of the compound obtained by the above reaction were as follows:

δ (ppm): 2.06 (m, 3H, CH₃), 5.49 (m, 1H, CH), 5.80 (m, 1H, CH), 7.19 (m, 2H, 2CH), 7.59 (m, 3H, 2CH, NH)

From the above ¹H-NMR results, it was confirmed that the compound obtained by the above reaction had a structure of the reaction product shown in the above reaction scheme.

### Example 5

### —Synthesis of Monomer 5

11.51 g (64.6 mmol) of 3-trifluoromethoxyphenol and 90 mL of dichloromethane were mixed in a four-neck flask and immersed and cooled in ice water. After adding 9.81 g (96.9 mmol) of triethylamine dropwise, 7.02 g (77.5 mmol) of acryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 14.67 g of the desired product.

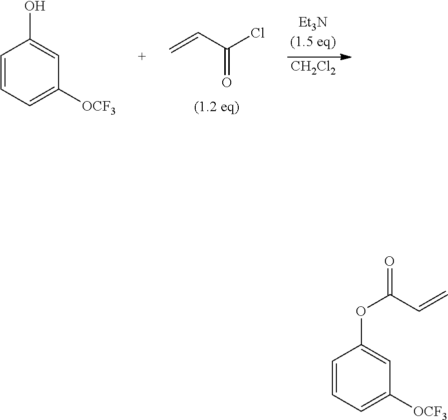

The ¹H-NMR chemical shifts of the compound obtained by the above reaction were as follows:

δ (ppm): 6.05 (dd, 1H, CH), 6.32 (dd, 1H, CH), 6.63 (dd, 1H, CH), 7.06 (m, 1H, CH), 7.12 (m, 2H, 2CH), 7.41 (t, 1H, CH)

From the above ¹H-NMR results, it was confirmed that the compound obtained by the above reaction had a structure of the reaction product shown in the above reaction scheme.

### Example 6

### —Synthesis of Monomer 6

10.85 g (60.9 mmol) of 3-trifluoromethoxyphenol and 90 mL of dichloromethane were mixed in a four-neck flask and immersed and cooled in ice water. After adding 9.25 g (91.4 mmol) of triethylamine dropwise, 8.60 g (82.3 mmol) of methacryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 13.71 g of the desired product.

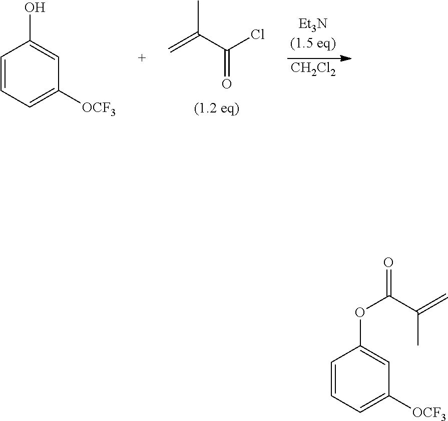

The ¹H-NMR chemical shifts of the compound obtained by the above reaction were as follows:

δ (ppm): 2.05 (m, 3H, CH₃), 5.78 (m, 1H, CH), 6.35 (m, 1H, CH), 7.04 (m, 1H, CH), 7.10 (m, 2H, 2CH), 7.40 (t, 1H, CH)

From the above ¹H-NMR results, it was confirmed that the compound obtained by the above reaction had a structure of the reaction product shown in the above reaction scheme.

### Example 7

### —Synthesis of Monomer 7

9.65 g (54.5 mmol) of 3-trifluoromethoxyaniline and 96 mL of dichloromethane were mixed in a four-neck flask and immersed and cooled in ice water. After adding 7.88 g (77.9 mmol) of triethylamine dropwise, 4.70 g (51.9 mmol) of acryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 11.44 g of the desired product.

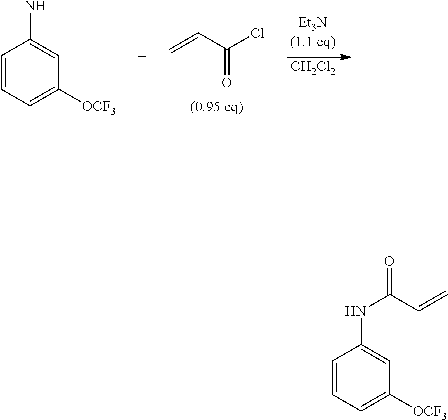

The ¹H-NMR chemical shifts of the compound obtained by the above reaction were as follows:

δ (ppm): 5.80 (dd, 1H, CH), 6.25 (dd, 1H, CH), 6.45 (dd, 1H, CH), 6.98 (dd, 1H, CH), 7.33 (t, 1H, CH), 7.44 (d, 1H, CH), 7.57 (brs, 1H, NH), 7.64 (s, 1H, CH) From the above ¹H-NMR results, it was confirmed that the compound obtained by the above reaction had a structure of the reaction product shown in the above reaction scheme.

### Example 8

### —Synthesis of Monomer 8

9.86 g (55.7 mmol) of 3-trifluoromethoxyaniline and 104 mL of dichloromethane were mixed in a four-neck flask and immersed and cooled in ice water. After adding 8.05 g (79.5 mmol) of triethylamine dropwise, 7.20 g (68.8 mmol) of methacryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 13.85 g of the desired product.

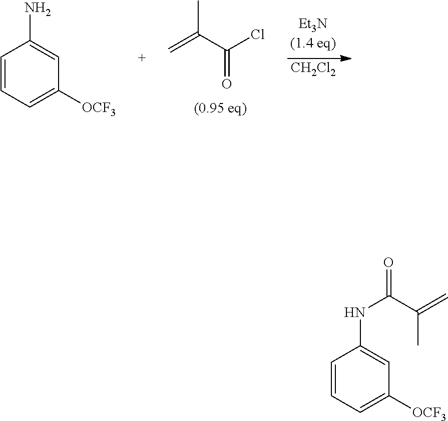

The ¹H-NMR chemical shifts of the compound obtained by the above reaction were as follows:

δ (ppm): 2.06 (m, 3H, CH₃), 5.50 (m, 1H, CH), 5.80 (m, 1H, CH), 6.98 (m, 1H, CH), 7.33 (t, 1H, CH), 7.42 (m, 1H, CH), 7.62 (m, 2H, CH, NH)

From the above ¹H-NMR results, it was confirmed that the compound obtained by the above reaction had a structure of the reaction product shown in the above reaction scheme.

### Example 9

### —Synthesis of Monomer 9

11.51 g (64.6 mmol) of 2-(trifluoromethoxy) phenol and 120 mL of dichloromethane were mixed in a four-neck flask and immersed and cooled in ice water. After adding 9.81 g (96.9 mmol) of triethylamine dropwise, 8.18 g (90.3 mmol) of acryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 14.04 g of the desired product.

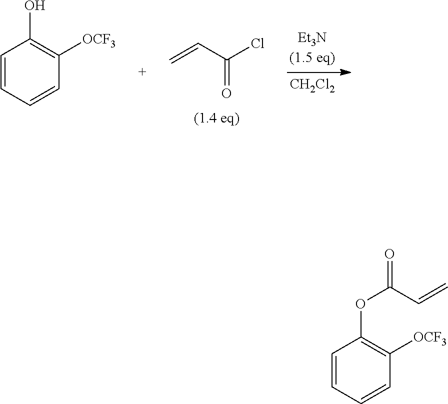

The ¹H-NMR chemical shifts of the compound obtained by the above reaction were as follows:

δ (ppm): 6.07 (dd, 1H, CH), 6.34 (dd, 1H, CH), 6.65 (dd, 1H, CH), 7.31 (m, 4H, 4CH)

From the above ¹H-NMR results, it was confirmed that the compound obtained by the above reaction had a structure of the reaction product shown in the above reaction scheme.

### Example 10

### —Synthesis of Monomer 10

10.85 g (60.9 mmol) of 2-(trifluoromethoxy) phenol and 90 mL of dichloromethane were mixed in a four-neck flask and immersed and cooled in ice water. After adding 9.25 g (91.4 mmol) of triethylamine dropwise, 8.92 g (85.4 mmol) of methacryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 14.85 g of the desired product.

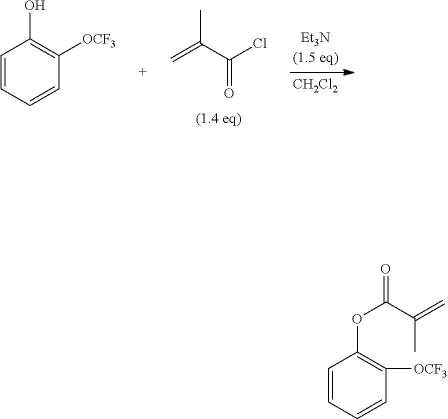

The ¹H-NMR chemical shifts of the compound obtained by the above reaction were as follows:

δ (ppm): 2.07 (m, 3H, CH₃), 5.80 (m, 1H, CH), 6.38 (m, 1H, CH), 7.30 (m, 4H, 4CH)

From the above ¹H-NMR results, it was confirmed that the compound obtained by the above reaction had a structure of the reaction product shown in the above reaction scheme.

### Example 11

### —Synthesis of Monomer 11

12.07 g (68.1 mmol) of 2-trifluoromethoxyaniline and 120 mL of dichloromethane were mixed in a four-neck flask and immersed and cooled in ice water. After adding 9.85 g (97.3 mmol) of triethylamine dropwise, 7.04 g (77.8 mmol) of acryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 13.70 g of the desired product.

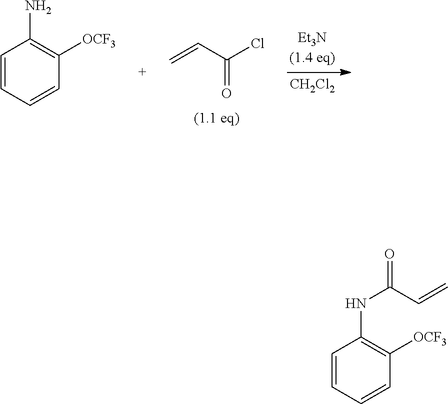

The ¹H-NMR chemical shifts of the compound obtained by the above reaction were as follows:

δ (ppm): 5.83 (dd, 1H, CH), 6.29 (dd, 1H, CH), 6.45 (dd, 1H, CH), 7.12 (m, 1H, CH), 7.28 (m, 2H, 2CH), 7.54 (brs, 1H, NH), 8.50 (d, 1H, CH)

From the above ¹H-NMR results, it was confirmed that the compound obtained by the above reaction had a structure of the reaction product shown in the above reaction scheme.

### Example 12

### —Synthesis of Monomer 12

12.73 g (71.9 mmol) of 2-trifluoromethoxyaniline and 120 mL of dichloromethane were mixed in a four-neck flask and immersed and cooled in ice water. After adding 10.80 g (106.7 mmol) of triethylamine dropwise, 10.57 g (101.1 mmol) of methacryloyl chloride was added dropwise, and the resulting mixture was allowed to raise the temperature to room temperature. After stirring overnight, hydrochloric acid (1 mol/L) was added to terminate the reaction, and the resulting mixture was separated into an aqueous layer and an organic layer. The obtained organic layer was washed using an aqueous sodium bicarbonate solution (2.5% by mass) and water, and the solvent was then distilled off under reduced pressure to obtain 13.74 g of the desired product.

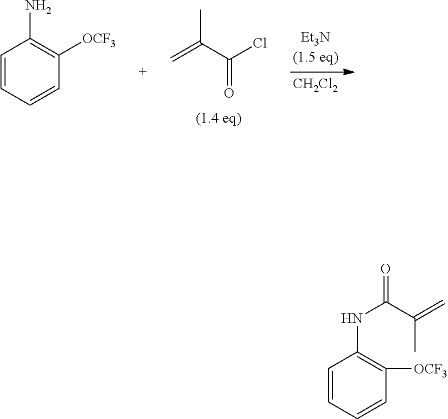

The ¹H-NMR chemical shifts of the compound obtained by the above reaction were as follows:

δ (ppm): 2.09 (m, 3H, CH₃), 5.53 (m, 1H, CH), 5.87 (m, 1H, CH), 7.13 (m, 1H, CH), 7.30 (m, 2H, 2CH), 7.90 (brs, 1H, NH), 8.49 (dd, 1H, CH) From the above ¹H-NMR results, it was confirmed that the compound obtained by the above reaction had a structure of the reaction product shown in the above reaction scheme.

### Example 13

### —Synthesis of Monomer 13

10.00 g (56.2 mmol) of 4-trifluoromethoxyphenol and 56 mL of tetrahydrofuran were mixed in a four-neck flask, and 0.36 g (2.81 mmol) of N,N-diisopropylethylamine and 8.72 g (61.8 mmol) of 2-isocyanatoethyl acrylate were added thereto, and the resulting mixture was heated to reflux for 21 hours. The desired product was then separated by column chromatography (hexane/acetone), and the solvent was distilled off under reduced pressure to obtain 16.10 g of the desired product. The yield of the obtained desired product was 89.8%.

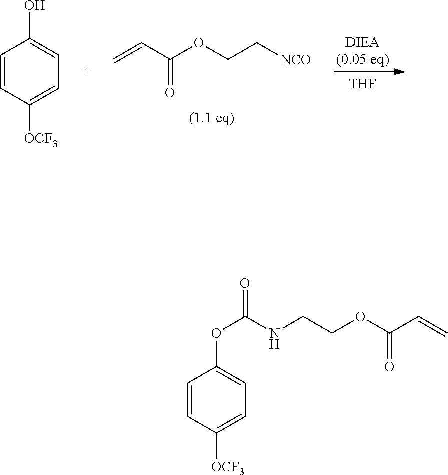

The ¹H-NMR chemical shifts of the compound obtained by the above reaction were as follows:

δ (ppm): 3.58 (m, 2H, CH₂), 4.31 (m, 2H, CH₂), 5.38 (brs, 1H, NH), 5.89 (dd, 1H, CH), 6.15 (dd, 1H, CH), 6.46 (dd, 1H, CH), 7.17 (m, 4H, 4CH) From the above ¹H-NMR results, it was confirmed that the compound obtained by the above reaction had a structure of the reaction product shown in the above reaction scheme.

### Example 14

### —Synthesis of Monomer 14

8.00 g (45.2 mmol) of 4-trifluoromethoxyaniline and 45 mL of tetrahydrofuran were mixed in a four-neck flask, and 7.01 g (49.68 mmol) of 2-isocyanatoethyl acrylate was added thereto, and the resulting mixture was heated to reflux for 14 hours. The desired product was then separated by column chromatography (hexane/ethyl acetate), and the solvent was distilled off under reduced pressure to obtain 10.80 g of the desired product. The yield of the obtained desired product was 74.9%.

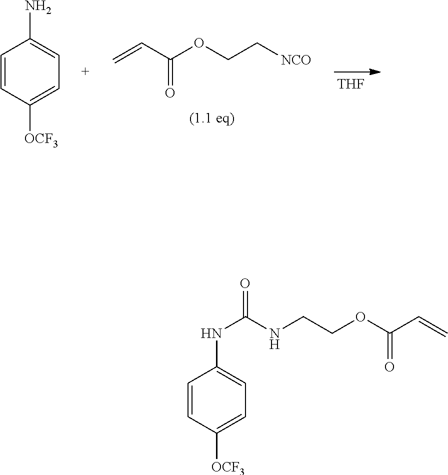

The ¹H-NMR chemical shifts of the compound obtained by the above reaction were as follows:

δ (ppm): 3.51 (t, 2H, CH₂), 4.24 (t, 2H, CH₂), 5.81 (m, 2H, CH, NH), 6.08 (dd, 1H, CH), 6.39 (dd, 1H, CH), 7.07 (m, 2H, 2CH), 7.26 (m, 2H, 2CH), 7.53 (m, 1H, NH)

From the above ¹H-NMR results, it was confirmed that the compound obtained by the above reaction had a structure of the reaction product shown in the above reaction scheme.

### Example 15

### —Synthesis of Precursor 15-1

First, 5.56 g (30.9 mmol) of 4-acetoxybenzoic acid and 56 mL of tetrahydrofuran were mixed in a four-neck flask, 4.01 g (33.7 mmol) of thionyl chloride was added dropwise thereto, and the resulting mixture was then heated to reflux. The disappearance of the raw materials was confirmed by GC, followed by concentration under reduced pressure, 56 mL of tetrahydrofuran, 3.99 g (30.9 mmol) of diisopropylethylamine, and 5.00 g (28.1 mmol) of 4-trifluoromethoxyphenol were added thereto, and the resulting mixture was stirred overnight. After confirming the disappearance of the raw materials by GC (gas chromatography), water was added to terminate the reaction, and after extraction with ethyl acetate, the organic layer was dried over magnesium sulfate. The obtained organic layer was separated by silica gel column chromatography (hexane/ethyl acetate) and concentrated under reduced pressure. The obtained crystals were recrystallized using methylene chloride and hexane, and the crystals were dried under reduced pressure to obtain 4.12 g of a precursor 15-1. The reaction scheme for obtaining the precursor 15-1 is as follows.

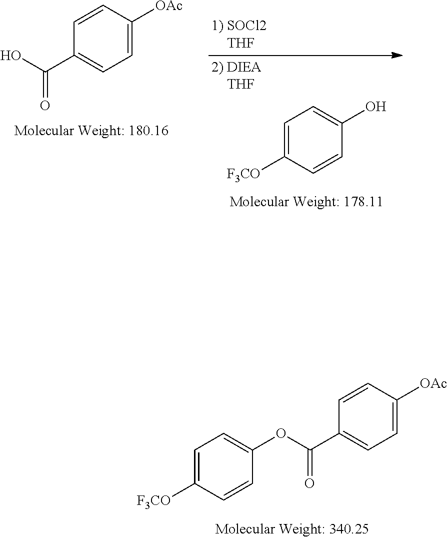 [SMILES: CC(=O)Oc1ccc(C(=O)O)cc1 ; Oc1ccc(OC(F)(F)F)cc1 ; CC(=O)Oc1ccc(C(=O)Oc2ccc(OC(F)(F)F)cc2)cc1]

### —Synthesis of Precursor 15-2

Then, 4.12 g (12.1 mmol) of the precursor 15-1 synthesized as described above was dissolved in a mixed solvent of 24 mL of tetrahydrofuran and 24 mL of methanol in a four-neck flask, and a 1:1 mixed solution of an aqueous ammonium chloride solution (1 mol/L) and an aqueous sodium hydroxide solution (1 mol/L) was added dropwise. The disappearance of the raw materials was confirmed by TLC, followed by neutralization with hydrochloric acid (1 mol/L) and extraction using methylene chloride. The obtained organic layer was concentrated under reduced pressure to obtain 3.72 g of a precursor 15-2 as a white solid. The reaction scheme for obtaining the precursor 15-2 is as follows.

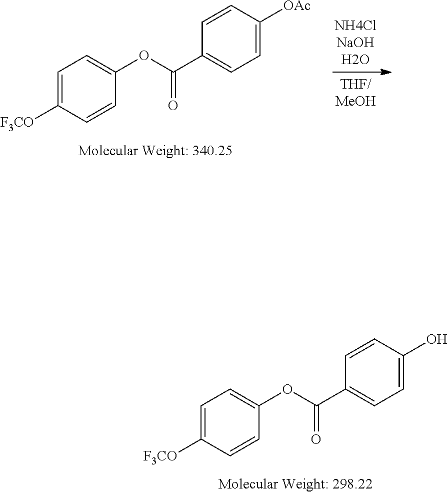 [SMILES: CC(=O)Oc1ccc(C(=O)Oc2ccc(OC(F)(F)F)cc2)cc1 ; O=C(Oc1ccc(OC(F)(F)F)cc1)c1ccc(O)cc1]

### —Synthesis of Monomer 15

3.72 g (12.5 mmol) of the precursor 15-2 synthesized as described above was dissolved in 25 mL of methylene chloride in a four-neck flask, and 1.77 g (13.7 mmol) of diisopropylethylamine was added dropwise. Subsequently, 1.24 g (13.7 mmol) of acryloyl chloride was added thereto, and the resulting mixture was stirred at room temperature for 2 hours. After confirming the disappearance of the raw materials by GC, water was added to terminate the reaction, and after extraction with methylene chloride, the organic layer was dried over magnesium sulfate. The obtained organic layer was separated by silica gel column chromatography (hexane/methylene chloride) and concentrated under reduced pressure. The obtained crystals were washed with hexane, and the crystals were dried under reduced pressure to obtain 2.48 g of the desired product.

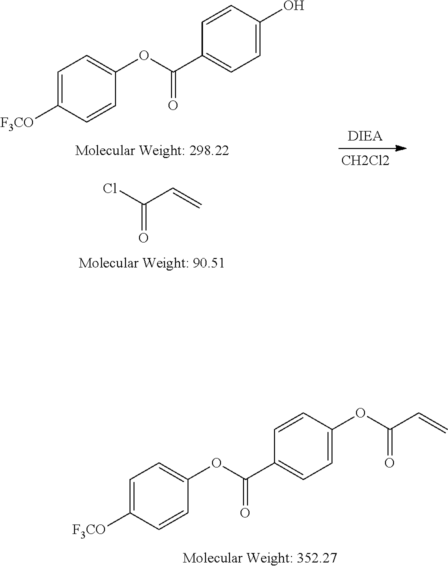

The ¹H-NMR chemical shifts of the compound obtained by the above reaction were as follows:

δ (ppm): 6.08 (d, 1H, CH), 6.35 (dd, 1H, CH), 6.66 (d, 1H, CH), 7.28 (m, 6H, 6CH), 8.25 (m, 2H, 2CH)

From the above ¹H-NMR results, it was confirmed that the compound obtained by the above reaction had a structure of the reaction product shown in the above reaction scheme.

### [Production of Fluorine-Containing Polymer]

### —Production of Fluorine-Containing Polymer 1

A reaction vessel was charged with the monomer 1 (100 parts) synthesized in Example 1 as described above, ethyl acetate (300 parts), and V-601 (1 part, dimethyl 2,2′-azobis(2-methylpropionate), manufactured by FUJIFILM Wako Pure Chemical Corporation), purged with nitrogen, and then sealed. A polymerization reaction was carried out at 70° C. for 18 hours to obtain an ethyl acetate solution containing a fluorine-containing polymer 1 as a polymer of the monomer 1. The reaction scheme for the above polymerization reaction is as follows.

A portion of the reaction liquid after 18 hours was collected, and the complete disappearance of the peak derived from the monomer 1 was confirmed by gas chromatography, thereby judging that the polymerization reaction had progressed quantitatively. The same confirmation by the reaction rate described above was also applied in other Examples.

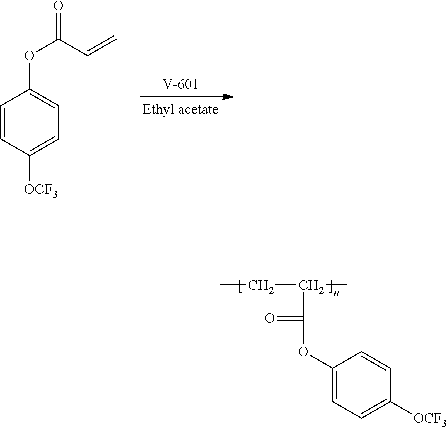

### —Production of Fluorine-Containing Polymers 2, 4 to 11, and 13 to 14

Fluorine-containing polymer 2 was produced by conducting polymerization in the same manner as in the production of the fluorine-containing polymer 1 described above, with the exception that 100 parts of the monomer 1 was changed to 100 parts of the monomer 2.

The same applies to the fluorine-containing polymers 4 to 11 and 13 to 14.

### —Production of Fluorine-Containing Polymer 3

A reaction vessel was charged with the monomer 3 (100 parts) synthesized in Example 3 as described above, ethyl acetate (700 parts), and V-601 (1 part, manufactured by FUJIFILM Wako Pure Chemical Corporation), purged with nitrogen, and then sealed. A polymerization reaction was carried out at 70° C. for 18 hours to obtain an ethyl acetate solution containing a fluorine-containing polymer 3. The reaction scheme for the above polymerization reaction is as follows.

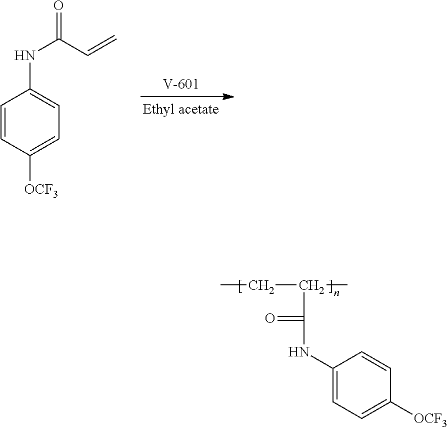

### —Production of Fluorine-Containing Polymer 12

A reaction vessel was charged with the monomer 12 (100 parts) synthesized in Example 12 as described above, ethyl acetate (400 parts), and V-601 (1 part, manufactured by FUJIFILM Wako Pure Chemical Corporation), purged with nitrogen, and then sealed. A polymerization reaction was carried out at 70° C. for 18 hours to obtain an ethyl acetate solution containing a fluorine-containing polymer 12. The reaction scheme for the above polymerization reaction is as follows.

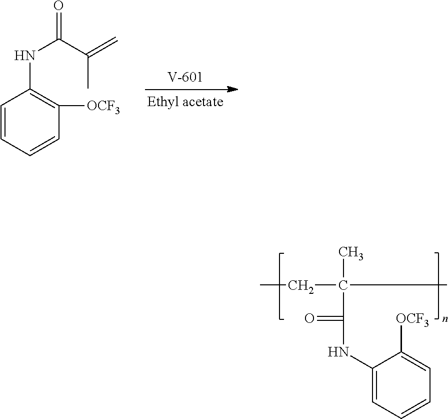

### —Production of Fluorine-Containing Polymer 15

A reaction vessel was charged with the monomer 15 (100 parts) synthesized in Example 15 as described above, ethyl acetate (500 parts), and V-601 (1 part, manufactured by FUJIFILM Wako Pure Chemical Corporation), purged with nitrogen, and then sealed. A polymerization reaction was carried out at 70° C. for 18 hours to obtain an ethyl acetate solution containing a fluorine-containing polymer 15. The reaction scheme for the above polymerization reaction is as follows.

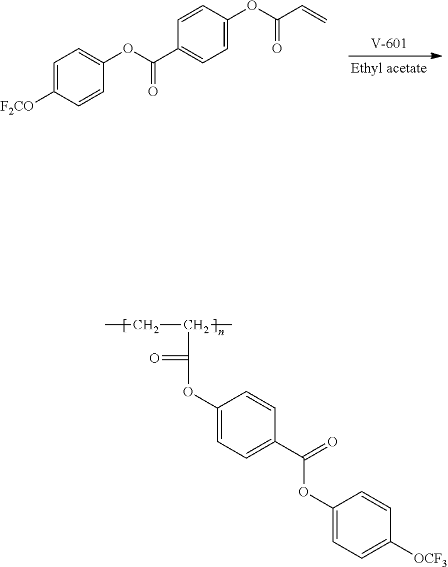

### Comparative Example 1

A reaction vessel was charged with phenyl acrylate (100 parts) as a monomer, ethyl acetate (300 parts), and V-601 (1 part, manufactured by FUJIFILM Wako Pure Chemical Corporation), purged with nitrogen, and then sealed. A polymerization reaction was carried out at 70° C. for 18 hours to obtain an ethyl acetate solution containing poly(phenyl acrylate). The reaction scheme for the above polymerization reaction is as follows.

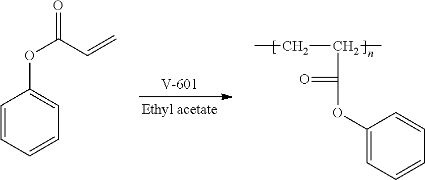

### Comparative Example 2

Polymerization was carried out in the same manner as in Comparative Example 1 as described above, with the exception that 100 parts of phenyl acrylate was changed to 100 parts of phenyl methacrylate, to obtain an ethyl acetate solution containing poly(phenyl methacrylate).

### Comparative Example 3

A reaction vessel was charged with N-phenylacrylamide (100 parts) as a monomer, ethyl acetate (700 parts), and V-601 (1 part, manufactured by FUJIFILM Wako Pure Chemical Corporation), purged with nitrogen, and then sealed. A polymerization reaction was carried out at 70° C. for 18 hours to obtain an ethyl acetate solution containing poly(N-phenylacrylamide). The reaction scheme for the above polymerization reaction is as follows.

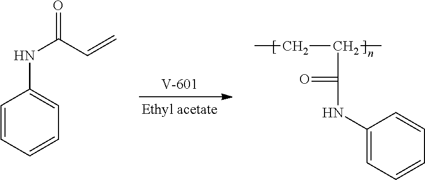

### Comparative Example 4

Polymerization was carried out in the same manner as in Comparative Example 1 as described above, with the exception that 100 parts of phenyl acrylate was changed to 100 parts of N-phenylmethacrylamide, to obtain an ethyl acetate solution containing poly(N-phenylmethacrylamide).

### [Production of Surface Treatment Agent]

Ethyl acetate was added to the respective solutions containing the polymers obtained as described above to produce surface treatment agents containing the polymers and solvent. For each surface treatment agent, the solid content concentration of the polymer in the surface treatment agent was 2% by mass.

### [Evaluation]

### <Preparation of Evaluation Specimens>

A glass plate was immersed in each of the surface treatment agents produced as described above for 1 minute. After pulling the glass plate out of the surface treatment agent, the glass plate was dried in a dryer at 120° C. for 5 minutes to obtain an evaluation specimen.

### <Measurement of Contact Angle>

Water or n-hexadecane (n-HD) was added dropwise onto the evaluation specimen obtained as described above, and the contact angle (unit: degrees) of water or n-hexadecane (n-HD) was measured using a contact angle meter DMo-501 (manufactured by Kyowa Interface Science Co., Ltd.). The contact angle was measured at five points respectively for water or n-hexadecane, and the average thereof was used as the evaluation value.

The evaluation results are shown in Table 1, together with the structural formula of each monomer.

### (Evaluation Criteria for Oil Repellency)

In the present disclosure, when the contact angle of n-hexadecane is 30 degrees or more, the oil repellency is judged to be excellent.

The greater the above contact angle in a range of 30 degrees or more, the superior the oil repellency.

It is preferred that the above contact angle is 40 degrees or more from the viewpoint of further improving oil repellency.

On the other hand, when the above contact angle is less than 30 degrees, the oil repellency is judged to be poor.

### (Evaluation Criteria for Water Repellency)

In the present specification, when the contact angle of water is 80 degrees or more, the water repellency is judged to be excellent.

The greater the above contact angle in a range of 80 degrees or more, the superior the water repellency.

It is preferred that the above contact angle is 100 degrees or more from the viewpoint of further improving water repellency.

On the other hand, when the above contact angle is less than 80 degrees, the water repellency is judged to be poor.

TABLE 1
Table 1 (Part 1)

| Example | | 1 | 2 | 3 | 4 |
|---|---|---|---|---|---|
| Monomer | | 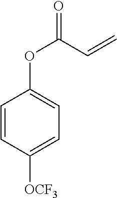 [SMILES: C=CC(=O)Oc1ccc(OC(F)(F)F)cc1] | 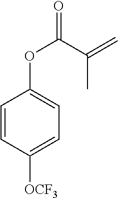 [SMILES: C=C(C)C(=O)Oc1ccc(OC(F)(F)F)cc1] | 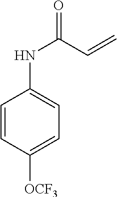 [SMILES: C=CC(=O)Nc1ccc(OC(F)(F)F)cc1] | 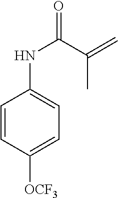 [SMILES: C=C(C)C(=O)Nc1ccc(OC(F)(F)F)cc1] |
| Contact | Water | 100 | 101 | 103 | 91 |
| angle | n-HD | 57 | 42 | 60 | 51 |
| (degree) | | | | | |
| Example | | 5 | 6 | 7 | 8 |
| Monomer | | 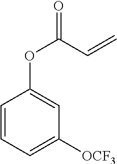 [SMILES: C=CC(=O)Oc1cccc(OC(F)(F)F)c1] | 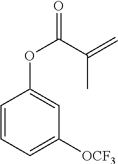 [SMILES: C=C(C)C(=O)Oc1cccc(OC(F)(F)F)c1] | 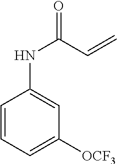 [SMILES: C=CC(=O)Nc1cccc(OC(F)(F)F)c1] | 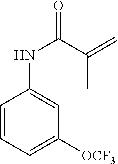 [SMILES: C=C(C)C(=O)Nc1cccc(OC(F)(F)F)c1] |
| Contact | Water | 95 | 93 | 97 | 100 |
| angle | n-HD | 46 | 36 | 53 | 41 |
| (degree) | | | | | |
| Example | | 9 | 10 | 11 | 12 |
| Monomer | | 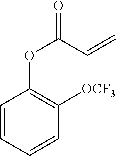 [SMILES: C=CC(=O)Oc1ccccc1OC(F)(F)F] | 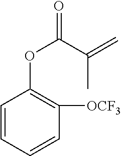 [SMILES: C=C(C)C(=O)Oc1ccccc1OC(F)(F)F] | 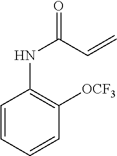 [SMILES: C=CC(=O)Nc1ccccc1OC(F)(F)F] | 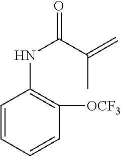 [SMILES: C=C(C)C(=O)Nc1ccccc1OC(F)(F)F] |
| Contact | Water | 88 | 87 | 93 | 95 |
| angle | n-HD | 34 | 34 | 31 | 38 |
| (degree) | | | | | |

| Example | | 13 | 14 | 15 |
|---|---|---|---|---|
| Monomer | | 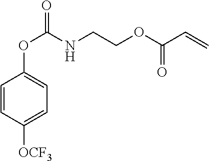 [SMILES: C=CC(=O)OCCNC(=O)Oc1ccc(OC(F)(F)F)cc1] | 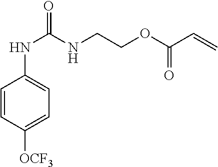 [SMILES: C=CC(=O)OCCNC(=O)Nc1ccc(OC(F)(F)F)cc1] | 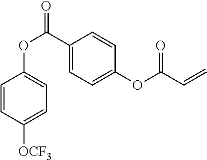 [SMILES: C=CC(=O)Oc1ccc(C(=O)Oc2ccc(OC(F)(F)F)cc2)cc1] |
| Contact | Water | 98 | 98 | 105 |
| angle | n-HD | 48 | 48 | 62 |
| (degree) | | | | |

TABLE 2
Table 1 (Part 2)

| Example | | 16 | 17 | 18 | 19 |
|---|---|---|---|---|---|
| Monomer | | 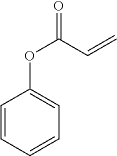 | 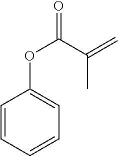 | 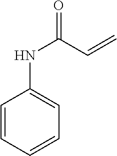 | 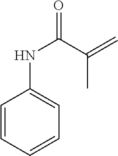 |
| Contact | Water | 90 | 87 | 83 | 79 |
| angle | n-HD | <10 | <10 | <10 | <10 |
| (degree) | | | | | |

From the results in Table 1, it was confirmed that the fluorine-containing polymers of the present disclosure, having structural units formed by the fluorine-containing compounds of the present disclosure, and the surface treatment agents of the present disclosure, exhibit the desired effects (Examples 1 to 15).

On the other hand, polymers and surface treatment agents having structural units formed by compounds without trifluoromethoxy groups exhibited poor oil repellency (Examples 16 to 19).

1. A fluorine-containing compound represented by formula (M) below: 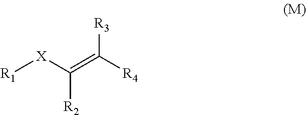
wherein X is an oxygen atom, a sulfur atom, or a linking group represented by formula (A) below; R₁ is a phenyl group in which at least one hydrogen atom is substituted with a trifluoromethoxy group; and each of R₂, R₃, and R₄ independently represents a hydrogen atom or an alkyl group having 1 to 4 carbon atoms, 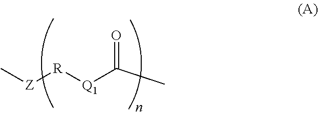
wherein n is an integer from 0 to 8; Q₁ is an oxygen atom or a divalent group represented by —NH—; R is a linear or branched alkylene group having 1 to 4 carbon atoms, or a phenylene group; and Y is any one of linking groups represented by formulas (y1) to (y4) below, 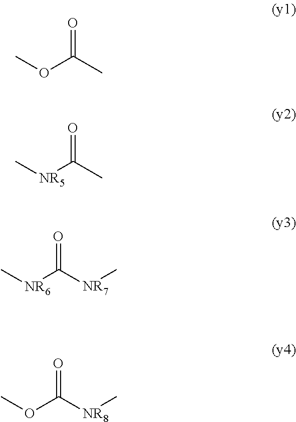
wherein each of R₅ and R₆ independently represents a hydrogen atom, an alkyl group having 1 to 4 carbon atoms, or a group represented by formula (B) below; and each of R₇ and R₈ independently represents a hydrogen atom or an alkyl group having 1 to 4 carbon atoms, 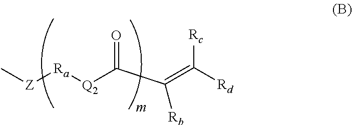
wherein m is an integer from 0 to 8; Q₂ is an oxygen atom or a divalent group represented by —NH—; Rₐ is a linear or branched alkylene group having 1 to 4 carbon atoms, or a phenylene group; each of R_{b}, R_{c}, and Rₐ independently represents a hydrogen atom or an alkyl group having 1 to 4 carbon atoms; and Z is a linking group represented by the following formula (z1) or (z2), 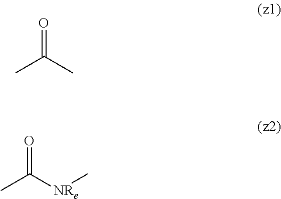
wherein Rₑ is a hydrogen atom or an alkyl group having 1 to 4 carbon atoms.

2. The fluorine-containing compound according to claim 1, wherein the trifluoromethoxy group in R₁ is bonded to a para position of the phenyl group.

3. The fluorine-containing compound according to claim 1, wherein X in the formula (M) is a linking group represented by the formula (A).

4. The fluorine-containing compound according to claim 3, wherein Y in the formula (A) is a linking group represented by the formula (y1) or (y2).

5. A fluorine-containing polymer comprising a structural unit formed by the fluorine-containing compound according to claim 1.

6. A surface treatment agent comprising the fluorine-containing polymer according to claim 5 and a solvent.
